(19) United States
(12) Patent Application Publication
(10) Pub. No.: US 2009/0221081 A1
(43) Pub. Date: Sep. 3, 2009

(54) METABOLITE MARKERS FOR WEIGHT MANAGEMENT

(76) Inventors: Steve M. Watkins, Sacramento, CA (US); Michelle M. Wiest, Moscow, ID (US)

Correspondence Address: Marshall, Gerstein & Borun LLP (TETHYS);233 S. Wacker Drive, Sears Tower, Suite 6300, Chicago, IL, 60606, US

(21) Appl. No.: 12/324,017

(22) Filed: Nov. 26, 2008

Related U.S. Application Data
(63) Continuation of application No. 11/226,616, filed on Sep. 13, 2005, now abandoned.
(60) Provisional application No. 60/609,703, filed on Sep. 13, 2004.

(51) Int. Cl. G01N 33/92

(52) U.S. Cl. 436/71

(57) ABSTRACT

The present invention provides methods of using certain metabolite markers for predicting weight development or its related conditions of a subject. The present invention also provides compositions and kits useful for detecting metabolite markers of the present invention.

### CROSS-REFERENCE TO RELATED APPLICATIONS

This application claims the benefit of provisional application No. 60/609,703, filed Sep. 13, 2004 under 35 U.S.C. §119(e), which is incorporated herein by reference.

### FIELD OF THE INVENTION

This invention relates generally to the field of metabolic targets and markers, especially markers indicative of weight loss or gain and conditions associated therewith.

### BACKGROUND OF THE INVENTION

Weight control has become increasingly relevant and important in modern daily life. Millions of people go on various types of diet each year, e.g., Weight-watcher, Jenny-Craig, NutriSystem, SlimFast, Atkins diet, The New Beverly Hill Diet, Liquid diet, The Pritikin Principle diet, The South Beach diet, etc. Many people become susceptible to disease conditions because of their weight gain while certain disease conditions cause weight loss. Metabolites and metabolic pathways are closely associated with weight and its related conditions in mammals, e.g., humans. Therefore, there is a need in the field to identify metabolic markers and targets for weight management and conditions associated therewith.

### SUMMARY OF THE INVENTION

The present invention is based, in part, on the discovery that certain metabolites or metabolic pathways can be used as diagnostic or therapeutic markers for projecting weight development patterns and conditions associated therewith. Accordingly the present invention provides methods for predicting weight development patterns and compositions and kits useful for detecting metabolites associated with various weight conditions.

In one embodiment, the present invention provides a method for predicting weight loss or weight gain of a subject. The method comprises measuring the level of a first metabolite marker in a sample of a subject under a condition, wherein the first metabolite marker is selected from the group consisting of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, TG18:3n3, CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3 and wherein the level of the first metabolite marker is predicative of weight loss or weight gain of the subject. The method also comprises measuring the level of a second and/or third metabolite marker of the present invention.

In another embodiment, the present invention provides a kit. The kit comprises an instruction for predicting weight condition of a subject and an agent capable of detecting the level of a metabolite marker in a sample of the subject, wherein the metabolite marker is selected from the group consisting of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, TG18:3n3, CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3.

For the purposes of this application, the abbreviations listed below have the following meaning.

| Abbreviations | Full Name |
|---|---|
| CE | cholesterol ester |
| CL | cardiolipin |
| DAG, DG | diacylglycerol |
| dm | dimethoxy acetal (derived from plasmalogen) |
| FA | free fatty acid |
| FC | free cholseterol |
| LYPC, LY | lysophosphatidylcholine |
| MUFA | mono unsaturated fatty acid |
| PC | phosphatidylcholine |
| PE | phosphatidylethanolamine |
| PI | phosphatidylinositol |
| PL | total phospholipid |
| PS | phosphatidylserine |
| PUFA | polyunsaturated fatty acid |
| SFA | saturated fatty acid |
| SM, SP | sphingomyelin |
| t- | trans- |
| TAG, TG | triacylglycerol |
| TGMUFA: | mono unsaturated fatty acid in triacylglycerol |
| TGPUFA: | polyunsaturated fatty acid in triacylglycerol |
| LC | lipid class |

### BRIEF DESCRIPTION OF THE DRAWINGS

FIG. 1 is a graph showing a non-limiting example of a fitted model (deconstructed signatures) and absolute weight loss at week six.

FIG. 2A is a graph showing the predicted weight loss in ZDF rats treated with PPARs agonists (Lipomics Model 1).

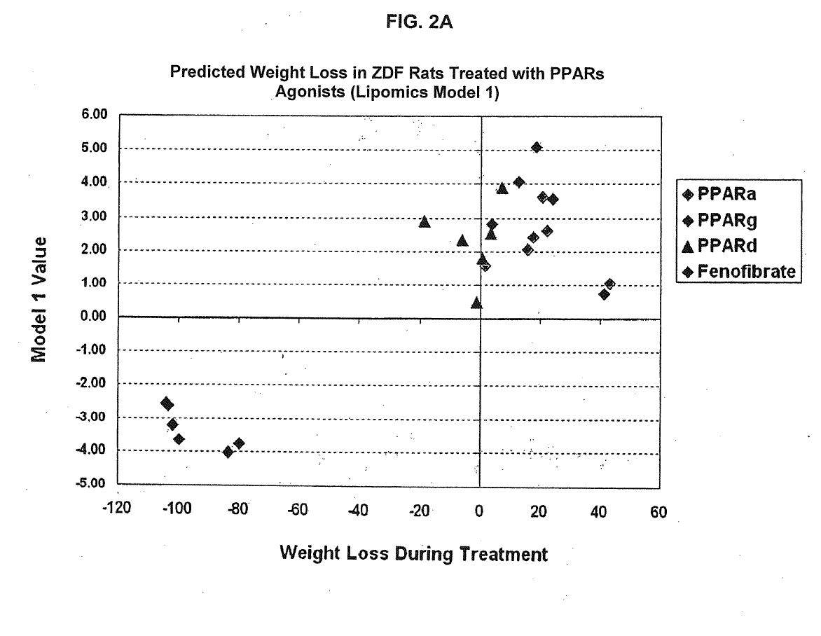

FIG. 2B is a graph showing the predicted weight loss in ZDF rats treated with PPARs agonists (Lipomics Model 2).

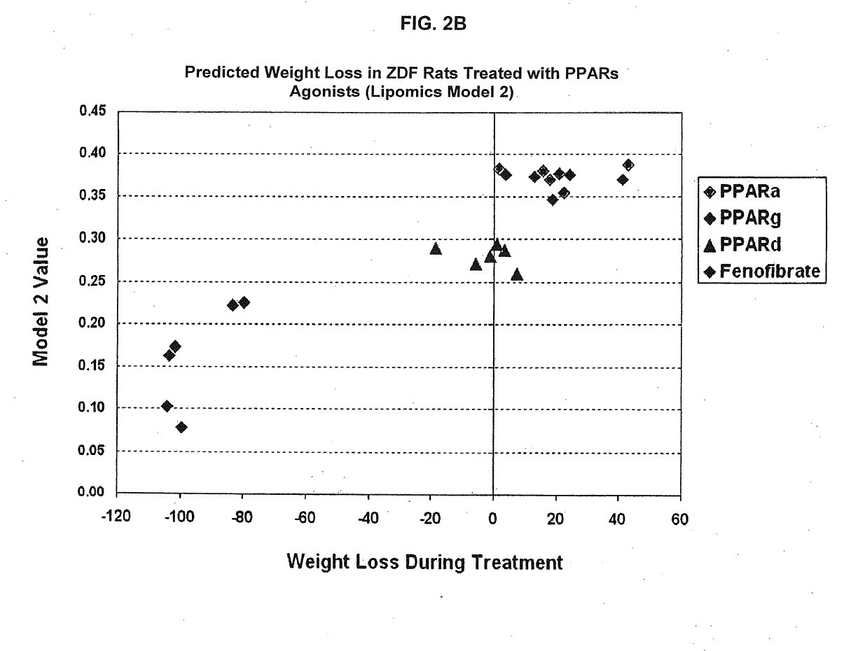

FIG. 2C is a graph showing the predicted weight loss in ZDF rats treated with PPARs agonists (Lipomics Model 3).

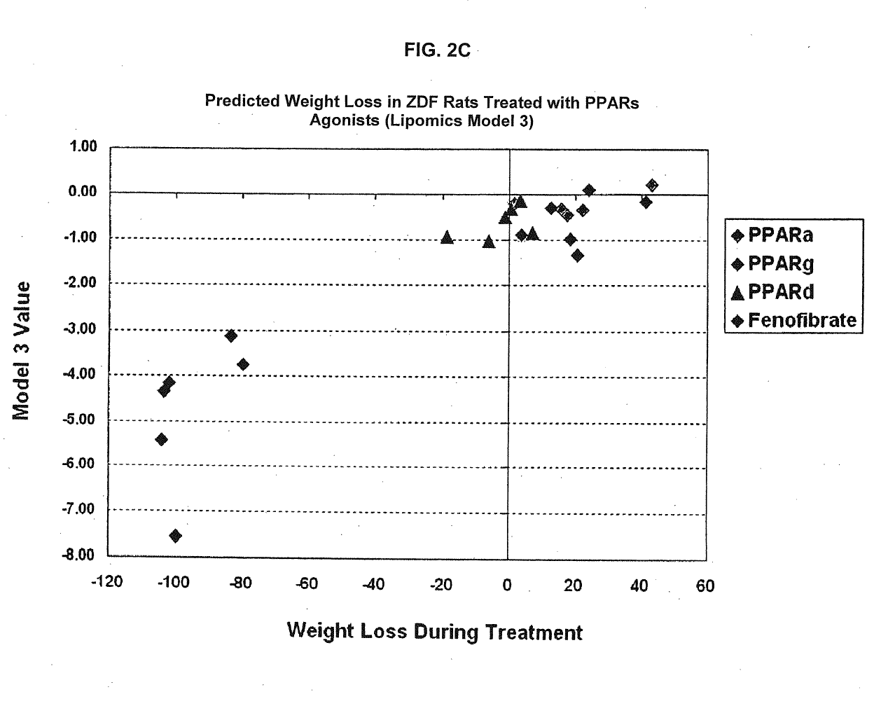

### DESCRIPTION OF THE PREFERRED EMBODIMENTS

The present invention is based, in part, on the discovery that certain metabolites or metabolic pathways can be used as diagnostic or therapeutic markers for projecting weight development patterns and conditions associated therewith. Accordingly, the present invention provides methods of using metabolite markers for predicting weight development patterns and conditions associated therewith, e.g., weight related conditions. In addition, the present invention also provides compositions and kits useful for detecting metabolites associated with weight loss or weight gain or weight related conditions.

According to the present invention, levels of certain metabolites can be used as markers for projecting weight or weight related conditions of a subject, e.g., human, animals or animal models for various disease conditions over a period of time. For example, levels of one or more metabolite markers can be compared to standard values of the metabolites corresponding to certain weight development patterns such as weight gain, weight loss, or weight neutral and the results of such comparison can be used to project weight development or weight related conditions of a subject over a period of time. In particular, a subject, e.g., human or animal having levels of certain metabolite markers similar to or within the standard values of these metabolites that are assigned to or associated with a weight loss pattern or weight loss related condition is likely to lose weight or develop the weight loss related condition over a period of time. Similarly a subject, e.g., human or animal having levels of certain metabolite markers similar to or within the standard values of these metabolites that are assigned to or associated with a weight gain or weight neutral pattern or their related conditions is likely to gain weight, experience no weight change, or develop weight gain related conditions over a period of time.

Standard values of metabolite markers assigned to different weight development conditions can be readily established for people with certain characteristics and/or undergoing certain conditions. For example, standard values of metabolites of interest can be established for people in certain age, gender, baseline body weight, and/or ethnic group, with certain physiological or pathological condition, and/or undergoing certain conditions, e.g., diet or therapeutic regimen. In general, one way of obtaining standard values of metabolite markers is by recording the levels of the metabolites from human or animals of interest and conducting standard statistical analyses, e.g., determining the average or mean value of the levels of such metabolites associated with a weight development pattern. Standard values of metabolite markers can also be similarly obtained for formulations containing one or more metabolites as variables.

According to the present invention, metabolite markers associated with the development of a weight or weight related condition, e.g., weight loss or weight gain or their related conditions include any, mono- or poly-, saturated or unsaturated free fatty acids or fatty acids linked to other molecules, e.g., via their carboxylic acid group. In one embodiment, metabolite markers of the present invention include fatty acids with long hydrocarbon tails, e.g., C14 to C24. In another embodiment, metabolite markers of the present invention include fatty acids of 14:0, 14:1n5, 16:0, 16:1n7, 18:0, 18:2n6, 18:1n7, 18:1n9, 18:3n3, 18:3n6, 20:2n6, 20:3n6, 20:3n9, 20:4n3, 20:4n6, 20:5n3, 22:5n3, 22:6n3, 24:1n9, n6, n7, n9, or lipid class. In yet another embodiment, metabolite markers of the present invention include mono unsaturated fatty acids, polyunsaturated fatty acids, and saturated fatty acids. In still yet another embodiment, metabolite markers of the present invention include fatty acids stored in cholesterol or as energy reserve, e.g., in triglycerides.

In general, levels of metabolite markers of the present invention can be levels of metabolite markers at a specific time point or over a period of time. For example, levels of metabolite markers of the present invention can be a baseline measurement of the metabolite markers. Such baseline measurement can be a measurement of the metabolite markers at the beginning of a condition or before any condition is imposed on a subject, e.g., diet or therapeutic regiment. In one embodiment, such baseline measurement is a measurement of metabolite markers at about day zero, day one, day two, or day three of a diet or therapeutic regiment.

Alternatively levels of metabolite markers of the present invention can be levels of metabolite markers over a period of time, e.g., a change of the level of a metabolite marker over a period of time. For example, the level of a metabolite marker of the present invention can be a change of such metabolite marker between a first time point and a second time point under a condition. In one embodiment, the level of a metabolite marker of the present invention is a change of such metabolite at the initial stage of a condition imposed to a subject, e.g., between about day one and day two, three, four, or day six of a diet or therapeutic regiment.

Levels of metabolite markers of the present invention can be expressed or characterized by various suitable means. For example, the level of a metabolite marker of the present invention can be either an absolute level or normalized level of the metabolite marker. In one embodiment, the level of a metabolite marker of the present invention is represented as 1) an absolute amount of the metabolite, e.g., concentration such as nanomoles per gram, etc., 2) the amount of the metabolite in a metabolite class, e.g. nanomoles per gram of a fatty acid as free fatty acid, in triacylglycerol, lysophophatidylcholine, phosphatidylethanolamine, or cholesterol ester, or 3) the amount of the metabolite relative to certain class of metabolites, e.g., mole percentage such as the amount of a fatty acid normalized against total fatty acid or the amount of a fatty acid in triacylglycerol normalized against total fatty acid in triacylglycerol or total fatty acid.

In another embodiment, the level of a metabolite marker of the present invention is normalized against another metabolite marker. For example, the level of a metabolite marker of the present invention can be a ratio between two or more metabolite markers of the present invention or can be normalized against an index metabolite marker associated with a pathway, enzymatic activity, class of metabolites, and/or status of certain metabolic activities. Alternatively the level of a metabolite marker of the present invention can be normalized against a housekeeping metabolite marker, e.g., the amount of which is relatively stable under one or more conditions imposed to a subject of interest.

Levels of metabolite markers of the present invention can be measured via any suitable means known or later developed in the art. For example, levels of metabolite markers of the present invention can be measured directly or indirectly by immunoassay, enzymatic assay, mass spectroscopy, fluorimetry, radioisotope detection, etc. Levels of metabolite markers of the present invention can also be measured via chromatography, e.g., gas chromatography, high performance chromatography nuclear magnetic resonance, thin-layer chromatography, etc. For example, levels of metabolite markers of the present invention can be measured according to the method described in Watkins et al., Journal of Lipid Research Vol. 43, pp 1-9, 2002 or methods described in PCT/US02/21426, both of which incorporated herein by reference.

Usually levels of metabolite markers of the present invention can be measured using any suitable biological sample of a subject, e.g., human. In one embodiment, such biological sample is a blood sample, e.g., plasma. In another embodiment, such biological sample is a tissue sample, e.g., adipose tissue, muscle, etc.

According to the present invention, levels of metabolite markers of the present invention can be either positively or negatively associated with weight gain (or weight loss) or its related conditions over a period of time under a condition, e.g., diet or therapeutic regiment. In general, positive association of a metabolite marker with weight gain or its related conditions means that a higher than a standard metabolite value assigned to weight gain or its related conditions is predictive of a level of weight gain higher than the level of weight gain corresponding to the standard metabolite value or development of weight gain related conditions. In other words, an increase in the level of a metabolite marker is associated or correlates with an increase in weight gain or development of weight gain related conditions in a subject.

Similarly a negative association of a metabolite marker with weight gain or its related conditions means that a higher than a standard metabolite value assigned to weight gain or its related conditions is predictive of a level of weight gain lower than the level of weight gain corresponding to the standard metabolite value, e.g., an increase in the level of a metabolite marker is associated or correlates with a decrease in weight gain or development of weight gain related conditions in a subject. Furthermore, a metabolite marker positively associated with weight gain or its related conditions normally can be negatively associated with weight loss or its related conditions, e.g., an increase in the level of a metabolite marker is associated or correlates with an increase in weight gain or development of weight gain related conditions and/or a decrease in weight loss in a subject.

In one embodiment, a baseline measurement of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, or TG18:3n3, or a combination thereof is positively associated with weight gain or its related conditions and/or negatively associated with weight loss or its related conditions while a baseline measurement of CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, or PC22:6n3, or a combination thereof is negatively associated with weight gain or its related conditions and/or positively associated with weight loss or its related conditions of a subject.

In another embodiment, a change of the level of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, or TG18:3n3, or a combination thereof over a period of time is positively associated with weight gain or its related conditions and/or negatively associated with weight loss or its related conditions while a change of the level of CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, or PC22:6n3, or a combination thereof over a period of time is negatively associated with weight gain or its related conditions and/or positively associated with weight loss or its related conditions of a subject.

According to the present invention, metabolite markers of the present invention can be used alone, in combination with each other, or in combination with other metabolites or factors in projecting weight development patterns of a subject. For example, metabolite markers of the present invention, especially metabolite markers that change concurrently or co-vary with each other under a condition, e.g., diet or therapeutic regiment can be used in connection with each other in projecting weight development or its related conditions of a subject. In one embodiment, at least one, two, three, four, five, or six metabolite markers of the present invention are used as a lipid panel in assessing weight development patterns or their related conditions of a subject, e.g., human or animal.

In another embodiment, metabolite markers like 16:1n7, e.g., in phosphatidylcholine, trans-phosphatidylcholine, triacylglycerol, trans-triacylglycerol, phosphatidylethanolamine, free fatty acid, or cholesterol ester and 18:1n9, e.g., in triacylglycerol, free fatty acid, cholesterol ester, or phosphatidylcholine dimethoxy acetal are used together for projecting weight development or its related conditions of a subject. In yet another embodiment, metabolite markers like polyunsaturated fatty acid, e.g., in triacylglycerol and 18:0, e.g., in triacylglycerol, free fatty acid, phophatidylcholine, lysophophatidylcholine, phosphatidylethanolamine dimethoxy acetal, or sphingomyelin are used together for projecting weight development or its related conditions of a subject.

In yet another embodiment, metabolite markers like 18:1n9, e.g., in triacylglycerol, free fatty acid, cholesterol ester, or phosphatidylcholine dimethoxy acetal and 18:0, e.g., in triacylglycerol, free fatty acid, phophatidylcholine, lysophophatidylcholine, phosphatidylethanolamine dimethoxy acetal, or sphingomyelin are used together for projecting weight development or its related markers of a subject. In still yet another embodiment, 16:1n7, e.g., in phosphatidylcholine, trans-phosphatidylcholine, triacylglycerol, trans-triacylglycerol, phosphatidylethanolamine, free fatty acid, or cholesterol ester and 18:2n6, e.g., in phosphatidylethanolamine, phosphatidylcholine, or cholesterol ester are used together for projecting weight development or its related markers of a subject.

In still another embodiment, metabolite markers like mono unsaturated fatty acid, e.g., in triacylglycerol or free fatty acid, 18:1n9, e.g. in triacylglycerol, free fatty acid, cholesterol ester, or phosphatidylcholine dimethoxy acetal and 18:0, e.g., in triacylglycerol, free fatty acid, phophatidylcholine, lysophophatidylcholine, phosphatidylethanolamine dimethoxy acetal, or sphingomyelin are used together to predict weight development or its related conditions of a subject. In still yet another embodiment, metabolite markers like 18:2n6, e.g., in phosphatidylethanolamine, phosphatidylcholine, or cholesterol ester, 18:1n9, e.g., in triacylglycerol, free fatty acid, cholesterol ester, or phosphatidylcholine dimethoxy acetal, and 16:0, e.g., in free fatty acid, phosphatidylcholine, lysophosphatidylcholine, or cholesterol ester are used together to predict weight development or its related conditions of a subject.

Metabolite markers of the present invention can also be used along with other characteristics of a subject in weight development or its related condition assessment, e.g., body weight, age, height, baseline body fat amount, etc. For example, metabolite markers of the present invention can be used in combination with body weight of a subject in projecting weight or its related conditions of the subject. The body weight can be baseline body weight or the body weight of a subject at a time point, e.g. after the initial stage of a diet or therapeutic regiment.

In one embodiment, metabolite markers like 18:0, e.g., in triacylglycerol, free fatty acid, phophatidylcholine, lysophophatidylcholine, phosphatidylethanolamine dimethoxy acetal, or sphingomyelin, or saturated fatty acid, e.g., in triacylglycerol are used in combination with the baseline body weight of a subject to predict weight development or its related conditions of the subject. In another embodiment, levels of metabolite markers like 18:0 in triacylglycerol, free fatty acid, phophatidylcholine, lysophophatidylcholine, phosphatidylethanolamine dimethoxy acetal, or sphingomyelin or saturated fatty acid in triacylglycerol are positively associated with weight loss or its related conditions of a subject when they are used in combination with the baseline body weight of the subject.

In yet another embodiment, levels of metabolite markers like 18:2n6, e.g., in phosphatidylethanolamine, phosphatidylcholine, or cholesterol ester, 18:1n9, e.g., in triacylglycerol, free fatty acid, cholesterol ester, or phosphatidylcholine dimethoxy acetal, and 16:0 in free fatty acid, phosphatidylcholine, lysophosphatidylcholine, or cholesterol ester are used in combination with the baseline body weight of a subject in projecting weight development or its related conditions of the subject.

According to the present invention, when analyzing the effects rendered by two or more metabolite markers of the present invention, one can either evaluate the effects of these metabolite markers individually or obtain the net effect of these metabolite markers, e.g., by using various mathematic formulas or models to quantify the effect of each metabolite marker. According to the present invention, a formula containing the levels of one or more metabolite markers of the present invention as variables includes any mathematic formula, model, equation, or expression established based on mathematic or statistical principles or methods using the values of one or more metabolite markers of the present invention as variables.

In general, any suitable mathematic analyses can be used to analyze the net effect of two or more metabolite markers of the present invention with respect to projecting the weight condition of a subject, e.g., human. For example, methods such as multivariate analysis of variance, multivariate regression, multiple regression can be used to determine relationships between dependent variables, e.g., weight loss or weight gain and independent variables, e.g., baseline body weight and levels of metabolite markers of the present invention. Clustering, including both hierarchical and nonhierarchical methods, as well as nonmetric Dimensional Scaling can be used to determine associations among variables and among changes in those variables.

In addition, principal component analysis is a common way of reducing the dimension of studies, and can be used to interpret the variance-covariance structure of a data set. Principal components may be used in such applications as multiple regression and cluster analysis. Factor analysis is used to describe the covariance by constructing “hidden” variables from the observed variables. Factor analysis may be considered an extension of principal component analysis, where principal component analysis is used as parameter estimation along with the maximum likelihood method. Furthermore, simple hypothesis such as equality of two vectors of means can be tested using Hotelling's T squared statistic.

In one embodiment, a formula containing one or more metabolite markers of the present invention as variables is established by using regression analyses, e.g., multiple linear regression. Examples of formulas developed based on multiple linear regression include, without any limitation, the following.

k+k₁(TG18:1n9)+k₂(FA18:1n9)+k₃(FA18:0)+k₄(CE16:1n7) Formula I

k+k₁(TGMUFA)+k₂(FA18:1n9)+k₃(FA18:0) Formula II

k+k₁(FA18:1n9)+k₂(FA18:0) Formula III

k+k₁(CE16:0)+k₂(TG18:1n9) Formula IV

k−k₁(TGPUFA)+k₂(FA18:1n9)+k₃(FA18:0)−k₄(TG18:0) Formula V

k+k₁(TG18:2n6)+k₂(TG18:1n9)+k₃(FA18:1n9)+k₄(FA18:0)+k₅(CE16:1n7) Formula VI

k+k₁(TGMUFA)+k₂(FA18:1n9)−k₃(CE16:0)+k₄(CE16:1n7) Formula VII

k−k₁(CE16:0)+k₂(CE16:1n7)+k₃(FA18:1n9)+k₄(TG18:1n9) Formula VIII

k+k₁(CE16:0)+k₂(CE16:1n7)+k₃(FA18:1n9)+k₄(TGMUFA) Formula IX

k+k₁(TG18:1n9)−k₂(CE16:0)+k₃(CE16:1n7)+k₄(FA18:1n9) Formula X

k+k₁(FA18:1n9)+k₂(TGMUFA)−k₃(CE16:0)+k₄(CE16:1n7) Formula XI

k+k₁(BW)−k₂(TG18:2n6)+k₃(FA18:1n9)−k₄(CE16:0) Formula XII

k, k₁, k₂, k₃, k₄, and k₅: constants

BW: baseline body weight

The constants of these formulas can be established by using a set of data obtained from known weight conditions. Usually the levels of metabolite markers used in these formulas can be either the levels at a time point or changes of levels over a period of time. For example, in Formulas I, II, III, IV, V, and VI the levels of metabolite markers used in these formulas are baseline measurements of these metabolite markers whereas in Formulas VII, VIII, IX, X, XI, and XII the levels of metabolite markers used in these formulas are changes of levels of these metabolite markers during the initial stage of a condition.

According to the present invention, mathematic formulas established using metabolite markers of the present invention can be used to either qualitatively or quantitatively assess the weight condition of a subject over a period of time. For example, a formula having one or more metabolite markers as variables can be used to directly calculate the weight condition of a subject. In addition, the net value of a formula containing one or more metabolite markers of the present invention can be compared to the standard value of such formula corresponding to a weight development pattern, e.g., weight loss, weight gain, or weight neutral and the results of such comparison can be used to project weight development. Specifically a subject, e.g., human having a net value of a formula similar to or within the range of the standard value of such formula that is assigned to or associated with a weight loss pattern or its related conditions is likely to lose weight or develop weight loss related conditions over a period of time. Similarly a subject, e.g., human having a net value of a formula similar to or within the range of the standard values of such formula that is assigned to or associated with a weight gain or weight gain related conditions or weight neutral pattern is likely to gain weight, develop weight gain related conditions, or experience no weight change over a period of time.

According to the present invention, weight related conditions, e.g., weight gain related conditions or weight loss related conditions include any physiological or pathological conditions associated with weight development or secondary to a weight condition. For example, weight gain can be associated with cardiovascular conditions, diabetic conditions, high blood pressure, stroke, sleep apnea, breast cancer, gall bladder, poor pregnancy outcome, kidney stones, arthritis, steatosis, steatohepatitis, colon cancer, endometrial cancer, etc whereas weight loss can be associated with depression, parasites, bulimia, anorexia, OTC drug side effects, AIDS/HIV, infection, TB, thyroid disease, chronic infections, GI diseases such as chronic IBS, periodontal disease, etc, wasting or neoplastic growth, e.g., cancer or tumor.

According to another aspect of the present invention, it provides compositions and kits useful for projecting weight conditions of a subject. In general such composition or kit contains an agent capable of detecting the levels of metabolite markers of the present invention and an instruction for using the level of metabolite markers of the present invention to predict weight loss. For example, the instruction can provide standard values corresponding to each weight development condition, e.g., formulas containing one or more metabolite markers of the present invention as variables or standard values for metabolites or formulas of the present invention.

The agent used in the kit of the present invention can be any agent useful for measuring the levels of metabolite markers of the present invention. In one embodiment, the agent contained in the kit of the present invention is an antibody, e.g., monoclonal antibody capable of detecting metabolite markers of the present invention. In another embodiment, the agent is a label modified to specifically identify metabolite markers of the present invention, e.g., a phosphorescent label, a fluorescent label, a biotin or avidin label, or a radioactive label. In yet another embodiment, the agent is a peptide or ligand containing a detectable moiety and capable of specifically binding to metabolite markers of the present invention. In still another embodiment, the agent is capable of reacting with the double bonds of unsaturated fatty acids to form various derivatives of double bonds, e.g., mercuric acetate derivatives, dimethyl disulfide addition, hydrogenation, deuteration, etc., which facilitate the measurements of the fatty acids.

According to the present invention, the methods and kits provided by the present invention can be used to assess weight conditions or development patterns under any condition for a subject, e.g., mammals such as humans or domesticated animals. Usually the methods and kits provided by the present invention can be used to predict weight conditions for people on a diet, e.g., to assess or predict the effectiveness of a dietary change or nutritional treatment. The methods and kits provided by the present invention can also be used to project weight development conditions for people undergoing or to undergo a therapeutic regiment, e.g., drug or hormonal treatments associated with weight conditions or the side effect of drugs or hormones on weight conditions. In one embodiment, metabolite markers of the present invention can be used to project susceptibility of weight related conditions, e.g., diabetic or cardiovascular conditions associated with weight gain or complications associated with weight loss or wasting.

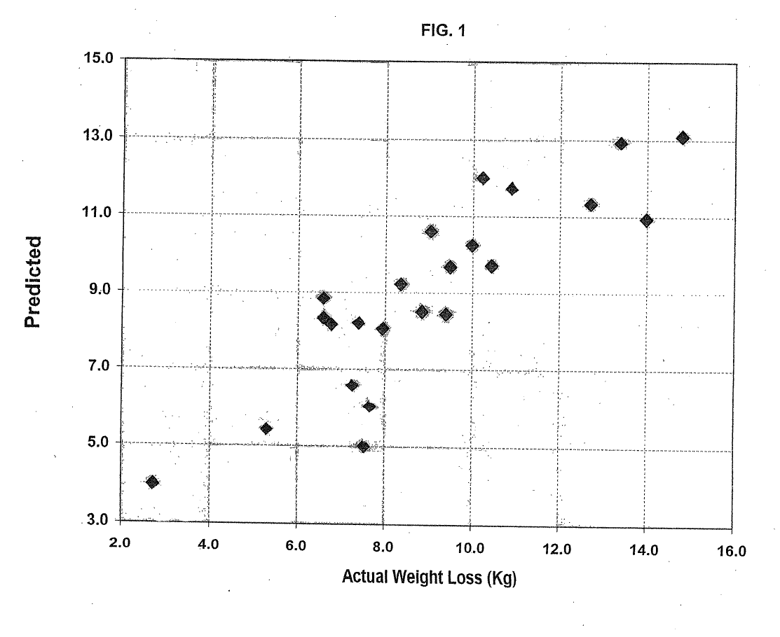

FIG. 1 shows a non-limiting example of a fitted model (deconstructed signatures) and absolute weight loss at week six. Additional information regarding this model is provided in Table 1, below:

TABLE 1
Model 4 Description (Individual Metabolites)

| Coefficients: | Estimate | Std. Error | t value | Pr(>\|t\|) |
|---|---|---|---|---|
| (Intercept) | 0.68734 | 1.89407 | 0.363 | 0.720915 |
| (Day 1 Body Weight) | 0.06531 | 0.01553 | 4.204 | 0.000534*** |
| (Δ Cholesterol ester | −1.01266 | 0.40443 | −2.504 | 0.022127* |
| 16:0₁₋₃) | | | | |
| (Δ Triglyceride | −0.37340 | 0.10557 | −3.537 | 0.002355** |
| 18:2n6₁₋₃) | | | | |
| (Δ Free fatty acid | | 0.08306 | 2.573 | 0.019175* |
| 18:1n9₁₋₃) | | | | |

Significance codes:
‘***’0.001,
‘**’0.01,
‘*’0.05
Residual standard error: 1.589 on 18 degrees of freedom
Multiple R-squared: 0.7477, Adjusted R-squared: 0.6917
F-statistic: 13.34 on 4 and 18 DF, p-value: 3.198e−05

Although the invention has been described with reference to the presently preferred embodiment, it should be understood that various modifications can be made without departing from the spirit of the invention. Accordingly, the invention is limited only by the following claims.

What is claimed is:

1. A method for predicting weight development of a subject comprising measuring the level of a first metabolite marker in a sample of a subject under a condition, wherein the first metabolite marker is selected from the group consisting of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, TG18:3n3, CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3, wherein the level of the first metabolite marker is predicative of weight development of the subject.

2. The method of claim 1, wherein the level of the first metabolite marker is a baseline measurement of the first metabolite marker.

3. The method of claim 1, wherein the level of the first metabolite marker is a change of the level of the first metabolite marker over a period of time.

4. The method of claim 1, wherein the level of the first metabolite marker is a baseline measurement of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, or TG18:3n3, and wherein a higher than a predetermined value of the first metabolite marker is predicative of a higher than a predetermined value of weight gain of the subject.

5. The method of claim 1, wherein the level of the first metabolite marker is a baseline measurement of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, or TG18:3n3, and wherein a higher than a predetermined value of the first metabolite marker is predicative of a lower than a predetermined value of weight loss of the subject.

6. The method of claim 1, wherein the level of the first metabolite marker is a baseline measurement of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, or TG18:3n3, and wherein a lower than a predetermined value of the first metabolite marker is predicative of a lower than a predetermined value of weight gain of the subject.

7. The method of claim 1, wherein the level of the first metabolite marker is a baseline measurement of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, or TG18:3n3, and wherein a lower than a predetermined value of the first metabolite marker is predicative of a higher than a predetermined value of weight loss of the subject.

8. The method of claim 1, wherein the level of the first metabolite marker is a baseline measurement of CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3, and wherein a lower than a predetermined value of the first metabolite marker is predicative of a higher than a predetermined value of weight gain of the subject.

9. The method of claim 1, wherein the level of the first metabolite marker is a baseline measurement of CE18:1n 9, PCdm18: n7, SM18:0, FA16: n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3, and wherein a lower than a predetermined value of the first metabolite marker is predicative of a lower than a predetermined value of weight loss of the subject

10. The method of claim 1, wherein the level of the first metabolite marker is a baseline measurement of CE18:1n 9, PCdm18: n7, SM18:0, FA16: n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3, and wherein a higher than a predetermined value of the first metabolite marker is predicative of a lower than a predetermined value of weight gain of the subject.

11. The method of claim 1, wherein the level of the first metabolite marker is a baseline measurement of CE18:1n 9, PCdm18: n7, SM18:0, FA16: n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3, and wherein a higher than a predetermined value of the first metabolite marker is predicative of a higher than a predetermined value of weight loss of the subject.

12. The method of claim 1, wherein the level of the first metabolite marker is measured directly or indirectly.

13. The method of claim 1, wherein measuring the level of the first metabolite marker includes chromatography, immunoassay, enzymatic assay, and mass spectroscopy.

14. The method of claim 1, wherein measuring the level of the first metabolite marker includes determining an end value of a formula containing the level of the first metabolite marker.

15. The method of claim 1, wherein the sample is blood, tissue, or plasma.

16. The method of claim 1, wherein the condition is prior to or during a diet session of the subject.

17. The method of claim 1, wherein the condition is prior to or during a therapeutic regiment administered to the subject.

18. The method of claim 1 further comprising measuring the body weight of the subject.

19. The method of claim 1 further comprising measuring the level of a second metabolite marker selected from the group consisting of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, TG18:3n3, CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3, wherein the first metabolite marker is different from the second metabolite marker.

20. The method of claim 19, wherein the level of the second metabolite marker is a baseline measurement of the second metabolite marker.

21. The method of claim 19, wherein the level of the second metabolite marker is a change of the level of the second metabolite marker over a period of time.

22. A method comprising measuring a lipid panel associated with a weight related condition, wherein the lipid panel comprises at least two metabolite markers selected from the group consisting of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, TG18:3n3, CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3.

23. A method comprising measuring a lipid panel associated with a weight related condition, wherein the lipid panel comprises at least three metabolite markers selected from the group consisting of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, TG18:3n3, CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3.

24. A method for assessing a weight related condition comprising measuring the level of a first metabolite marker in a sample of a subject under a condition, wherein the first metabolite marker is selected from the group consisting of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, TG18:3n3, CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3, wherein the level of the first metabolite marker is predictive of the weight related condition of the subject.

25. A method for assessing a weight related condition comprising measuring a lipid panel, wherein the lipid panel comprises at least two metabolite markers selected from the group consisting of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n 5, TGt16:1n7, CE18:3n3, PEdm18:0, LYLC, TG18:3n3, CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3.

26. A kit comprising an instruction for predicting weight condition of a subject and an agent capable of detecting the level of a metabolite marker in a sample of the subject, wherein the metabolite marker is selected from the group consisting of CE16:1n7, LY20:3n6, PC20:3n6, PE20:3n6, CE14:0, PC20:3n9, CELC, PCt16:1n7, TGSAT, TG14:0, TGn7, CE20:3n6, TG16:1n7, FA18:0, FASAT, FA14:0, FA16:0, PC14:0, TG18:0, CE18:3n6, PC20:2n6, PC20:4n3, PE18:2n6, TG20:3n6, CE20:4n3, PC18:2n6, PELC, PC18:3n6, PC20:5n3, PC18:3n3, PE20:5n3, PCLC, CE18:2n6, PC18:0, LY18:0, TGLC, PC16:1n7, PE16:1n7, FA14:1n5, TGt16:1n7, CE18:3n3, PEdm18:0, LYLC, TG18:3n3, CE18:1n9, PCdm18:1n7, SM18:0, FA16:1n7, PE20:4n6, FALC, CE18:1n7, PCn7, PC18:1n7, CEn9, FA18:1n7, PC16:0, LY16:0, TGMUFA, TGn6, FAn7, PCdm18:1n9, CESAT, CE16:0, SM24:1n9, TGn9, TG18:1n9, FAMUFA, TG22:5n3, TGPUFA, TG20:4n6, FA18:1n9, FAn9, PC20:4n6, CE20:4n6, TG22:6n3, and PC22:6n3.

27. The kit of claim 26, wherein the agent contains a detectable moiety.

28. The kit of claim 26, wherein the agent contains a moiety detectable by chromatography, immunoassay, enzymatic assay, or mass spectroscopy.

29. The kit of claim 26, wherein the instruction contains a formula containing the level of the metabolite marker as a variable.

30. The kit of claim 26, wherein the instruction contains a standard value of the metabolite marker for a weight development condition.
